(19) United States
(12) Patent Application Publication
(10) Pub. No.: US 2024/0329051 A1
(43) Pub. Date: Oct. 3, 2024

(54) PEPTIDE ANALYSIS METHOD AND PEPTIDE ANALYSIS APPARATUS

(71) Applicant: SHIMADZU CORPORATION, Kyoto (JP)

(72) Inventors: Eiichi MATSUO, Kyoto-shi (JP); Yoshihiro HAYAKAWA, Kyoto-shi (JP); Shinichi KAWANO, Kyoto-shi (JP); Kazuhiro IMAI, Kyoto-shi (JP)

(73) Assignee: SHIMADZU CORPORATION, Kyoto (JP)

(21) Appl. No.: 18/293,546

(22) PCT Filed: Aug. 6, 2021
(86) PCT No.: PCT/JP2021/029358

§ 371 Date: Jun. 7, 2024

(51) Int. Cl. G01N 33/68; G01N 1/40; G01N 33/58

(52) U.S. Cl. CPC G01N 33/6848; G01N 1/4044; G01N 33/58; G01N 33/6815

(57) ABSTRACT

The present invention includes: preparing a sample containing a peptide to be analyzed; adding a labeling compound which specifically reacts with a thiol group to the sample, and labeling a reduced cysteine residue contained in the peptide in the sample with the labeling compound; separating a sample containing the peptide labeled with the labeling compound by a chromatograph; executing product ion scan measurement with a peptide ion, as a precursor ion, generated by ionizing the peptide labeled with the labeling compound under a condition that a thiol group is not desorbed from a cysteine residue; and determining whether or not the peptide to be analyzed contains a reduced cysteine residue by determining whether or not a peak appearing at a specific mass-to-charge ratio corresponding to a structure of the labeling compound is present in a mass spectrum created based on data obtained by the measurement execution.

### TECHNICAL FIELD

The present invention relates to a peptide analysis method and a peptide analysis apparatus for determining whether or not an amino acid having a thiol group constituting a protein or a peptide is in a reduced form with a chromatograph mass spectrometer.

### BACKGROUND ART

Cysteine (Cys) is a sulfur-containing amino acid having a thiol group (SH group) in the side chain, and exists in many proteins. The SH group is highly reactive, and forms a disulfide (SS) bond with an SH group of another amino acid including cysteine, or the SS bond is broken to return to the SH group in a protein molecule. The number and position of SS bonds in the molecule of a protein determine the three-dimensional structure of the protein. In addition, Cys residues have been often used as a target of a specific reaction by a nucleophilic substitution reaction or nucleophilic addition reaction to a thiol group (Non Patent Literature 1).

Albumin, a type of protein contained in blood, contains 35 cysteine residues (Cys residues), and typically, among these 35 Cys residues, only the 34th Cys residue (hereinafter, referred to as “Cys 34”) from the N-terminal side has an SH group in a free state, and all the SH groups of the remaining 34 Cys residues form an SS bond in the molecule. However, a free SH group of one albumin in blood may form an SS bond with a free SH group of another albumin, or may form an SS bond with a free SH group contained in a protein or peptide other than albumin. Albumin in which the SH group of Cys 34 is in a free state is called reduced albumin, and albumin forming an SS bond is called oxidized albumin. In recent years, it has been reported that there is a correlation between the ratio of reduced albumin and oxidized albumin in blood and a specific disease, and it has been studied to use the ratio of reduced albumin and oxidized albumin as a marker for screening or diagnosis of the specific disease.

Conventionally, discrimination between oxidized albumin and reduced albumin is generally performed by liquid chromatography. However, there is no confirmation whether the separation by liquid chromatography is caused only by the difference in the redox state of the Cys residue.

To address this, there is a method for detecting a protein and/or peptide containing a Cys residue with DAABD-Cl(4-(dimethylaminoethyl aminosulfonyl)-7-chloro-2,1,3-benzoxadiazole), which is a fluorescent reagent specifically reacting with an SH group (Patent Literatures 1 and 2, Non Patent Literatures 2 to 4). In this method, in order to increase the detection sensitivity, a sample containing a protein and/or a peptide is subjected to a reduction treatment, one or a plurality of SS bonds contained in the protein and/or the peptide are cleaved to form SH groups, and then DAABD-Cl is added. As a result, the SH group of the Cys residue of the protein and/or peptide reacts with DAABD-Cl, and DAABD binds to the Cys residue to provide a fluorescently labeled protein and/or peptide. Therefore, the protein and/or peptide is separated by fluorescence detection, and if necessary, the protein and/or peptide is fragmented by trypsin digestion and then introduced into a mass spectrometer. Then, the amino acid sequence of each fragment is determined by collating data (MS spectrum, MS/MS spectrum) obtained by MS/MS analysis of the fragment of the fluorescently labeled protein and/or peptide with information stored in a database, and the protein and/or peptide is identified.

### CITATION LIST

### Patent Literature

Patent Literature 1: JP 2005-017264 A
Patent Literature 2: WO 2005/056146 A

### Non Patent Literature

Non Patent Literature 1: L. Dayon; Thiol-targeted Microspray Mass Spectrometry of Peptides and Proteins through On-line EC-tagging. PhD Thesis, Ecole Polytechnique Fédérale de Lausanne, 2006.
Non Patent Literature 2: T. Santa, T. Fukushima, T. Ichibangase, and K. Imai; Biomed. Chromatogr. 2008, 22 (4), pp. 343-353
Non Patent Literature 3: M. Masuda, C. Toriumi, T. Santa, and K. Imai; Anal. Chem. 2004, 76, pp. 728-735
Non Patent Literature 4: T. Ichibangase and K. Imai; J. Pharam. Biomed. Anal. 2014, 101, pp. 31-39

### SUMMARY OF INVENTION

### Technical Problem

In the above method, it is possible to selectively introduce a fluorescently labeled protein and/or peptide, that is, a protein and/or peptide having a Cys residue into the mass spectrometer, but from the result of MS/MS analysis of a fragment of the protein and/or peptide, it is not possible to determine whether or not the fragment is a fragment to which DAABD is bound (fluorescently labeled fragment). Therefore, it is necessary to determine the amino acid sequence of each fragment by collating MS/MS analysis data obtained for all fragments with information stored in the database.

Although it is conceivable to selectively detect a fluorescently labeled fragment by multiple reaction monitoring (MRM) measurement with a mass spectrometer, in this case, it is necessary to prepare a standard sample in which DAABD is bound to a Cys residue for peptides of various sequences, and measure these to provide MS/MS data of each standard sample. In addition, when analyzing the MS/MS data obtained for each standard sample, it is necessary to set a combination of mass-to-charge ratios (m/z) of precursor ions and product ions optimal for detection of a fluorescently labeled fragment and control parameters such as collision energy optimal for the combination, which is troublesome and complicated.

An object of the present invention is to provide a peptide analysis method and a peptide analysis apparatus capable of determining whether a Cys residue of a protein or a peptide is in a reduced form or an oxidized form without performing troublesome and complicated operations.

### Solution to Problem

The present invention made to solve the above problems is a peptide analysis method for analyzing a reduced form and an oxidized form of a cysteine residue contained in a peptide with a chromatograph mass spectrometer capable of MS/MS measurement, the peptide analysis method including:
a) a step of preparing a sample containing a peptide to be analyzed;
b) a step of adding a labeling compound which specifically reacts with a thiol group to the sample, and labeling a reduced cysteine residue contained in the peptide in the sample with the labeling compound;
c) a separation step of separating a sample containing the peptide labeled with the labeling compound by a chromatograph;
d) a measurement execution step of executing product ion scan measurement with a peptide ion, as a precursor ion, generated by ionizing the separated peptide labeled with the labeling compound under a condition that a thiol group is not desorbed from a cysteine residue; and
e) a determination step of determining whether or not the peptide to be analyzed contains a reduced cysteine residue by determining whether or not a peak appearing at a specific mass-to-charge ratio corresponding to the structure of the labeling compound is present in a mass spectrum created based on data obtained in the measurement execution step.

In addition, the present invention made to solve the above problems is a peptide analysis apparatus including a chromatograph mass spectrometer capable of MS/MS measurement and configured to analyze a reduced form and an oxidized form of a cysteine residue contained in a peptide, the peptide analysis apparatus including:
a) an analysis controller configured to operate the chromatograph mass spectrometer so as to separate a sample containing a peptide labeled with a labeling compound which specifically reacts with a thiol group by a chromatograph, and to perform product ion scan measurement with a peptide ion, as a precursor ion, generated by ionizing the separated sample under a condition that a thiol group is not desorbed from a cysteine residue;
b) a mass spectrum creation unit configured to create a mass spectrum based on data obtained by the product ion scan measurement; and
c) a determination unit configured to determine whether or not a peak appearing at a specific mass-to-charge ratio corresponding to the structure of the labeling compound is present in the mass spectrum to determine whether or not the peptide to be analyzed contains a reduced cysteine residue.

In the present invention, examples of the chromatograph constituting the chromatograph mass spectrometer capable of MS/MS measurement include a gas chromatograph, a liquid chromatograph, capillary electrophoresis, and the like, and examples of the mass spectrometer include a tandem quadrupole mass spectrometer (also referred to as triple quadrupole mass spectrometer), a quadrupole time-of-flight mass spectrometer (QTOF), an ion trap mass spectrometer, a matrix-assisted laser desorption/ionization mass spectrometer, and the like, and a combination thereof can be used.

In the present invention, the “peptide” includes both a protein and a peptide. In addition, the reduced cysteine residue (Cys residue) refers to one in which a thiol group (SH group) of the Cys residue is in a free state, and the oxidized Cys residue refers to one in which a disulfide bond (SS bond) is formed between the Cys residue and another Cys residue in the molecule of the same peptide, or one in which an SS bond is formed between a Cys residue of a certain peptide and a Cys residue of another peptide.

A peptide has a configuration in which a plurality of amino acids are peptide-bonded, and when the peptide in the sample contains a reduced Cys residue, an SH group of the Cys residue reacts with a labeling compound to provide a peptide in which the Cys residue is labeled with the labeling compound. Herein, the term “labeled with a labeling compound” refers to, for example, a state in which H (hydrogen) of the SH group and a part of the labeling compound are separated, and S (sulfur) of the SH group and the remaining part of the labeling compound are bonded to form a new functional group (hereinafter, also referred to as “labeled functional group”). In this case, H separated from the SH group and a part of the labeling compound are also bound to form a molecule.

In the present invention, a sample containing a peptide labeled with a labeling compound is separated by a chromatograph and then introduced into a mass spectrometer. At this time, when the molecular weight of the peptide to be analyzed is relatively small, the sample is introduced into the chromatograph as it is, but when the molecular weight is relatively large, the peptide labeled with the labeling compound may be digested with a protein digestion enzyme to be fragmented into a peptide having a small molecular weight, and then the peptide is introduced into the chromatograph. For convenience of explanation, hereinafter, a peptide labeled with a labeling compound is introduced into a mass spectrometer.

Under the conditions in the present invention, when a peptide in a sample is introduced into a mass spectrometer and ionized, a thiol group is not desorbed from a cysteine residue. Therefore, as the ion source of the mass spectrometer, it is preferable to use an ion source that performs what is called soft ionization such as electrospray ionization (ESI) or atmospheric pressure chemical ionization (APCI).

When ionized under the conditions described above and the peptide in the sample contains a reduced Cys residue, ions of the peptide in a state in which the Cys residue is labeled with a labeling compound (that is, a state of having a labeled functional group) are generated by ionization. When such a peptide ion is dissociated by collision dissociation or the like, the labeled functional group is desorbed. Therefore, when the peptide ion is subjected to the product ion scan measurement as a precursor ion, a peak appears on the MS/MS spectrum of the product ion at a specific mass-to-charge ratio corresponding to the structure of the labeled functional group, that is, the structure of the labeling compound. Therefore, if the presence of such a peak is confirmed, it can be determined that the Cys residue contained in the peptide in the sample is highly likely to be in a reduced form.

On the other hand, when no peak is detected at the specific mass-to-charge ratio, it can be determined that the Cys residue contained in the peptide in the sample is in an oxidized form or that no Cys residue is highly likely to be contained in the peptide in the sample. In this case, whether or not the peptide in the sample contains a Cys residue can be determined by identifying the peptide by collating the MS/MS spectrum with information stored in the database. Even when a peak is detected in the specific mass-to-charge ratio, the peptide can be identified by collating a pattern obtained by removing the peak from the MS/MS spectrum with information stored in the database.

In the present invention, a compound represented by the following formula (1) can be used as the labeling compound.

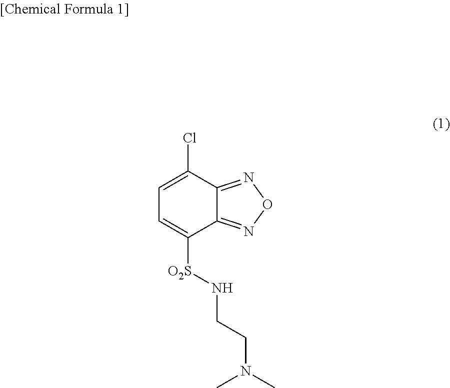 [SMILES: CN(C)CCNS(=O)(=O)c1ccc(Cl)c2nonc12]

This compound is a DAABD-Cl reagent known as a reagent that fluorescently labels a peptide by specifically binding to an SH group, and examples of specific mass-to-charge ratios corresponding to the structure of the labeling compound in this case include “303.06” and “344.08”.

It is considered that a peak having a mass-to-charge ratio of 303.06 corresponds to an ion represented by the following formula (2), and a peak having a mass-to-charge ratio (m/z) of 344.08 corresponds to an ion represented by the following formula (3). Each of the peaks corresponds to an ion derived from DAABD-Cl.

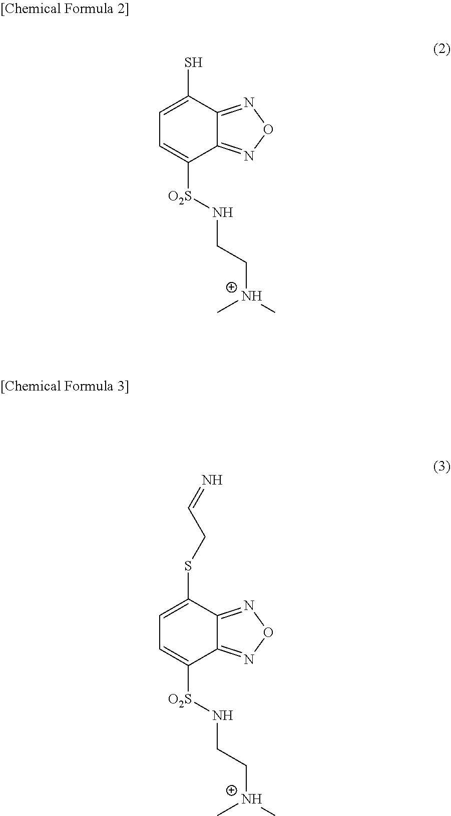 [SMILES: C[NH+](C)CCNS(=O)(=O)c1ccc(S)c2nonc12 ; C[NH+](C)CCNS(=O)(=O)c1ccc(SCC=N)c2nonc12]

### Advantageous Effects of Invention

According to the present invention, it is possible to estimate whether or not a peptide contains a reduced Cys residue by determining whether or not a peak having a specific mass-to-charge ratio corresponding to a structure of a labeling compound which specifically reacts with an SH group is present in a mass spectrum created based on data obtained by adding a labeling compound to a sample containing a peptide to be analyzed and then analyzing the sample using a chromatograph mass spectrometer. Therefore, it is possible to eliminate the need for troublesome and complicated work such as identifying a peptide by collating data obtained for the sample with information stored in a database, or setting conditions and the like for measuring the sample by actually measuring a standard sample.

### BRIEF DESCRIPTION OF DRAWINGS

FIG. 1 is a schematic configuration diagram of one embodiment of a peptide analysis apparatus for performing a peptide analysis method according to the present invention.

FIG. 2 is a flowchart showing a procedure of peptide analysis processing.

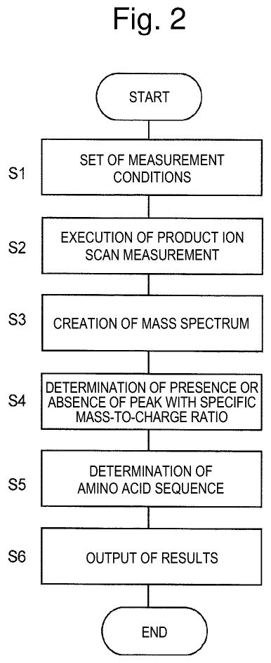

FIG. 3 is a diagram showing an example of a result of performing MS/MS ion search on a result of LC-MS/MS analysis of a sample containing human albumin.

FIG. 4 is a diagram showing an MS/MS spectrum with serial number: 571 and an amino acid sequence identified from the spectrum.

FIG. 5 is a diagram showing an MS/MS spectrum with serial number: 589 and an amino acid sequence identified from the spectrum.

FIG. 6 is a diagram showing an MS/MS spectrum with serial number: 627 and an amino acid sequence identified from the spectrum.

FIG. 7 is a diagram showing an example of an MS/MS spectrum with serial number: 655 and an amino acid sequence identified from the spectrum.

FIG. 8 is a diagram showing a result of MS/MS ion search of a sample containing a peptide in which no peak was observed in a mass-to-charge ratio corresponding to a structure of DAABD-Cl.

FIG. 9 is an example of an MS spectrum and an MS/MS spectrum of a sample containing glutathione.

FIG. 10 is an example of an MS spectrum and an MS/MS spectrum of a sample containing oxytocin.

FIG. 11 is a diagram showing peaks having a common mass-to-charge ratio appearing in MS/MS spectra of samples each containing albumin, glutathione, and oxytocin.

### DESCRIPTION OF EMBODIMENTS

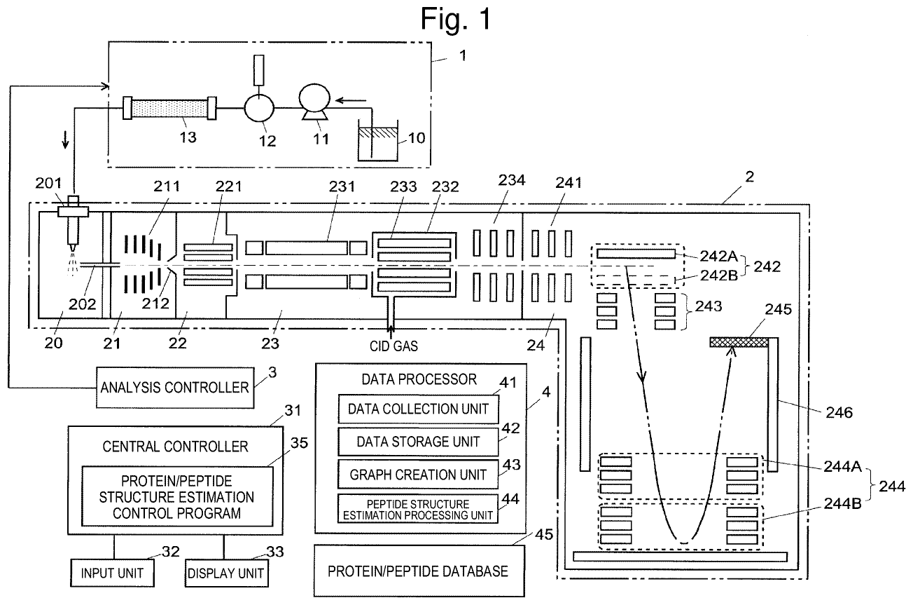

Hereinafter, an analysis method according to the present invention will be described with reference to the drawings. FIG. 1 is a schematic configuration diagram of a peptide analysis apparatus for performing a peptide analysis method according to the present invention. This peptide analysis apparatus is a liquid chromatograph system (LC-MS/MS system) including a liquid chromatograph (LC) unit 1 and a mass spectrometry (MS/MS) unit 2.

The LC unit 1 includes a mobile phase container 10 in which a mobile phase is stored, a pump 11 for drawing the mobile phase and supplying the mobile phase at a fixed flow rate, an injector 12 for injecting a predetermined amount of sample liquid into the mobile phase, and a column 13 for temporally separating various compounds contained in the sample liquid.

The MS/MS unit 2 has the configuration of a multi-stage differential pumping system including an ionization chamber 20 maintained at approximately atmospheric pressure and an analysis chamber 24 evacuated to a high degree of vacuum by a vacuum pump (not shown), between which first, second, and third intermediate chambers 21, 22 and 23 are provided having their degrees of vacuum increased in a stepwise manner. The ionization chamber 20 is provided with an electrospray ionization probe (ESI probe) 201 to nebulize a sample liquid eluted from the column 13 of the LC unit 1, while imparting electric charges to the solution.

The ionization chamber 20 communicates with the first intermediate chamber 21 through a heated thin capillary 202. The first intermediate chamber 21 is separated from the second intermediate chamber 22 by a skimmer 212 having a small hole at its apex. The first and second intermediate chambers 21 and 22 respectively contain ion guides 211 and 221 for transporting ions to the next stage while converging the ions. The third intermediate chamber 23 contains a quadrupole mass filter 231 for separating ions according to their mass-to-charge ratios, a collision cell 232 containing a multipole ion guide 233, and an ion guide 234 for transporting ions ejected from the collision cell 232. A CID gas, such as argon or nitrogen, is continuously or intermittently supplied into the collision cell 232.

The analysis chamber 24 contains: an ion transport electrode 241 for receiving ions from the third intermediate chamber 23 and transporting them to the orthogonal accelerator section; an orthogonal acceleration electrode 242 including two electrodes 242A and 242B arranged in such a manner as to face each other across the axis of incidence of the ions (orthogonal acceleration area); an acceleration electrode 243 for accelerating ions ejected into the flight space by the orthogonal acceleration electrode 242; a reflectron electrode 244 (244A and 244B) for forming a return path for the ions within the flight space; a detector 245; and a flight tube 246 located on the outer edge of the flight space.

In the MS/MS unit 2, an MS scan measurement, MS/MS scan measurement, or MSⁿ scan measurement (where n is an integer equal to or greater than three) can be performed. For example, in the case of the MS/MS scan measurement (product ion scan measurement), only an ion designated as the precursor ion is allowed to pass through the quadrupole mass filter 231. Additionally, a CID gas is supplied into the collision cell 232 to fragment the precursor ion into product ions. The product ions are introduced into the flight space, and the mass-to-charge ratios of the ions are determined based on their respective times of flight.

An analysis controller 3 has the function of controlling the operation of each of the LC unit 1 and the MS/MS unit 2 under the command of a central controller 31. The central controller 31, which has an input unit 32 and a display unit 33 connected, is responsible for providing a user interface through these units and for conducting a general control of the entire system. A storage device included in the central controller 31 stores a protein/peptide structure estimation control program 35 that performs characteristic control for estimating the structure or amino acid sequence of a specific amino acid (specifically, cysteine) residue included in a protein or peptide to be analyzed. A CPU or the like controls each unit through the analysis controller 3 according to the program 35, thereby executing measurement and data processing necessary for estimating the structure of the peptide.

At this time, a detection signal (ionic intensity signal) by the detector 245 is input to a data processor 4. The data processor 4 includes a data collection unit 41, a data storage unit 42, a graph creation unit 43, and a peptide structure estimation processing unit 44 as functional blocks, and estimates the structure and amino acid sequence of cysteine contained in the peptide by executing data processing using the information stored in a protein/peptide database 45, and determines whether the protein or peptide in the sample has a reduced Cys residue or identifies the protein or peptide from these results. As will be described later, the determination of whether or not the Cys residue is in the reduced form depends on whether or not a peak of a specific mass-to-charge ratio appears in the MS/MS spectrum. The mass-to-charge ratio of such peaks is determined previously, and thus may be stored in the protein/peptide structure estimation control program 35.

In the protein/peptide database 45, time-of-flight-vs-mass-to-charge-ratio information and applied-voltage information are stored. Time-of-flight-vs-mass-to-charge-ratio information is a set of information describing the length of time required for each of the ions with various mass-to-charge ratios to fly in the flight space in the mass spectrometer unit 2. Applied-voltage information is a set of information concerning the values of the voltages applied to the ion transport electrode 241, orthogonal acceleration electrode 242, acceleration electrode 243, reflectron electrode 244, and flight tube 246. In the present embodiment, a plurality of different levels of the applied voltage depending on the ion-ejection period is related to the orthogonal acceleration electrode 242.

The central controller 31 and the data processor 4 can be embodied by using a personal computer as hardware and executing a dedicated controlling and processing software program installed in that computer. In this case, a keyboard and a pointing device (mouse) serve as the input unit 32, and a display monitor serves as the display unit 33.

When the structure of the Cys residue contained in the peptide is analyzed using the LC-MS/MS system described above, first, a sample containing the peptide to be analyzed is prepared. At this time, a labeling compound which specifically reacts with the thiol group is added to the sample, and the thiol group of the Cys residue contained in the peptide in the sample is reacted with the labeling compound. When such pretreatment is completed, the measurer sets the sample in the LC-MS/MS system, and operates the input unit 32 to set conditions such as a separation condition in the LC unit 1, an ionization condition in the MS/MS unit 2, and an MS/MS measurement condition (step S1).

When the measurement is started according to the instruction of the measurer, the analysis controller 3 controls the LC unit 1 and the MS/MS unit 2 based on the instruction from the central controller 31. As a result, the sample separated in the LC unit 1 is introduced into the MS/MS unit 2, ionized, and then subjected to product ion scan measurement (step S2). At this time, the ionization is performed under the condition that the labeling compound is not separated from the peptide. Data obtained by the product ion scan measurement is stored in the data storage unit 42 in the data processor 4.

When the measurement is completed, the graph creation unit 43 processes the data to create a mass spectrum (step S3). Then, a peak is extracted from the mass spectrum, and it is determined whether or not a peak having a specific mass-to-charge ratio is included in the extracted peak (step S4). In addition, the amino acid sequence of the peptide is estimated by collating peak information of the mass spectrum with information stored in the protein/peptide database (step S5). When the above processing is completed, the determination result of step S4 and the estimation result of the amino acid sequence are output to the display unit 33 (step S6). The determination result of step S4 is, for example, the number of reduced Cys residues contained in the peptide in the sample, the ratio between the reduced Cys residues and the oxidized Cys residues, and positions of the reduced Cys residues in the amino acid sequence.

Then, an actual measurement example by the LC-MS/MS system according to the above-described embodiments will be described, and it will be shown that the system is useful for determining whether the Cys residue contained in the protein or peptide is a reduced form or an oxidized form.

### 1. Measurement Example of Human Albumin

### (1) DAABD Modification of Human Albumin

The following solution and buffer solution were added to 10 μL of human albumin (1 mM), and finally water was added and mixed so that the total amount was 100 μL, then the mixture was gently stirred at 500 rpm for 10 minutes at 40° C., and human albumin and DAABD-Cl were reacted. Then, 3 μL of a 20% TFA aqueous solution was added to the mixture after stirring to terminate the reaction.
CHAPS aqueous solution of 3.3 mM EDTA/17 mM: 58.8 μL
TCEP (42 mM) aqueous solution: 1.2 μL
Guanidine hydrochloride (GdnHCl) buffer of 6 M (pH 8.7): 25 μL
DAABD-Cl acetonitrile solution of 0.14 M: 5 μL

EDTA is an abbreviation of “ethylenediaminetetraacetic acid”, CHAPS is “3-[(3-cholamidopropyl)dimethylammonio]propanesulfonate”, TCEP is “tris(2-carboxyethyl)phosphine hydrochloride”, and TFA is “trifluoroacetic acid”. The structural formula of DAABD-Cl is shown below.

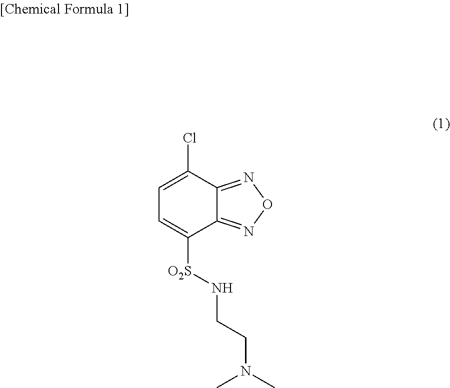 [SMILES: CN(C)CCNS(=O)(=O)c1ccc(Cl)c2nonc12]

### (2) Fractionation by High Performance Chromatography (HPLC)

From 103 μL of the reaction solution obtained in the above (1), 10 μL (about 1 nmol of human albumin) was collected, and supplied into a preparative column to fractionate an albumin fraction (about 2 to 5 mL). The fractionated albumin fraction was dried to solid in a centrifugal dryer (or a freeze dryer). LC conditions are shown below.

### <LC Conditions>

HPLC: Nexera XR manufactured by Shimadzu Corporation

Column: Aeris WIDEPORE 3.6 u XB-C8 (ODS group-modified silica gel packed HPLC column (inner diameter: 4.6 mm, length: 250 mm, particle diameter of filler: 3.6 μm)) manufactured by Phenomenex Corporation

Column temperature: 60° C.

Elution method: Gradient elution with two liquids of mobile phase A: 0.1% TFA, 1% IPA, 9% ACN aqueous solution, and mobile phase B: 0.1% TFA, 1% IPA, 74% ACN aqueous solution

Mobile phase flow rate: 0.8 mL/min

### (3) Reduction Treatment

The dried and solidified albumin fraction obtained in the above (2) was dissolved in 75 μL of a 500 mM Tris HCl (pH 8.0) aqueous solution containing urea (8 M). To this was added and mixed 1 μL of TCEP aqueous solution (42 mM) and the mixture was warmed at 37° C. for 30 minutes.

An alkylation treatment is typically performed after the reduction treatment, but this treatment is not performed in the actual measurement example presented herein. The reason is that, herein, the protein is first subjected to reduction treatment and then DAABD modification, and thus all Cys residues reduced to free SH groups are DAABD modified. However, if necessary depending on the purpose or the analysis method, the alkylation treatment is performed as appropriate. As a method of the alkylation treatment, 1 μL of IAA (iodoacetamide) (100 mM) is further added to the mixed solution and mixed, and the mixture is heated at 37° C. for 30 minutes.

### (4) Trypsin Digestion

To the mixed solution warmed to 37° C. in (3), 225 μL of a 50 mM NH₄CO₃ aqueous solution was added and mixed to provide 300 μL of a mixed sample liquid, and then trypsin digestion was performed. Trypsin digestion was performed using a trypsin column (MonoSpin (registered trademark) Trypsin column (manufactured by GL Sciences Inc.)). Specifically, first, the trypsin column was equilibrated with a 50 mM NH₄CO₃ aqueous solution, and the mixed sample liquid was gently passed therethrough twice to perform trypsin digestion. The mixed sample liquid after trypsin digestion is referred to as trypsin digestion sample liquid.

### (5) LCMS/MS Measurement

In 300 μL of the trypsin digestion sample liquid obtained in the above (4), 3 μL (corresponding to 10 pmol of human albumin) was introduced into HPLC (Nexera X2 manufactured by Shimadzu Corporation) and subjected to LC separation, followed by LCMS analysis and LCMS/MS analysis using LCMS-IT-TOF (manufactured by Shimadzu Corporation). Herein, MS/MS (auto) analysis was performed by selecting three peaks in descending order of intensity from among peaks detected in a mass spectrum obtained by LCMS analysis.

LC: Nexera XR manufactured by Shimadzu Corporation

Column: XR-ODS III (ODS group-modified silica gel packed HPLC column (inner diameter: 2.0 mm, length: 150 mm, particle diameter of filler: 2.2 μm)) manufactured by Shimadzu Corporation

Column temperature: 40° C.

Elution method: Gradient elution with 2 liquids of mobile phase A: 5 mM ammonium formate/0.1% formic acid aqueous solution, and mobile phase B: 0.1% formic acid acetonitrile solution

Mobile phase flow rate: 0.3 mL/min

Ionization: ESI

Ion accumulation: 30 msec

Mass range: MS m/z 150-1,500, MS/MS m/z 50-2,000

### (6) Database Search

The peak information extracted from the MS/MS spectrum obtained by the MS/MS analysis in the above (5) was subjected to MS/MS ion search mounted on Mascot manufactured by Matrix Science, thereby identifying peptides corresponding to each peak on the mass spectrum and also identifying the assignment of fragments corresponding to each peak on the MS/MS spectrum.

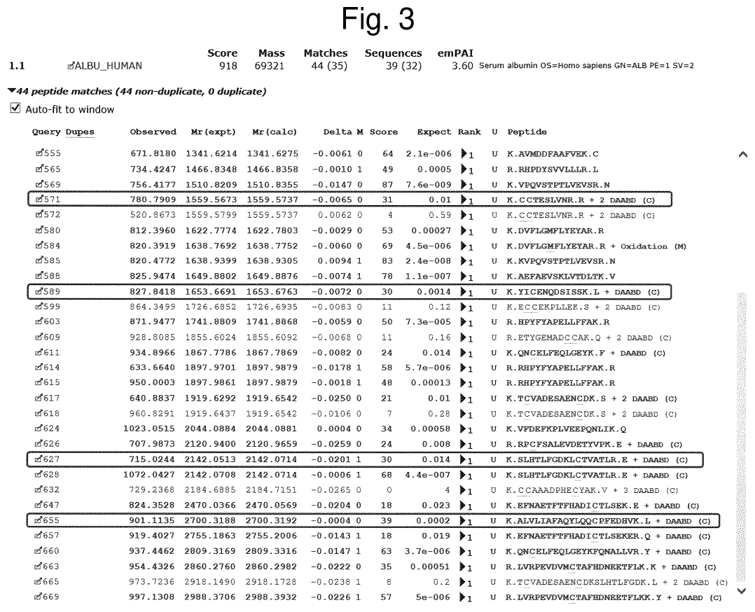

FIG. 3 shows a part of the results of the MS/MS ion search. This indicates information on peptide identification of each MS/MS spectrum, and in order from the left, the serial number of the MS/MS spectrum data (Query), the experimental value of the m/z value of the precursor ion (Observed), the experimental value of the mass of the precursor molecule (Mr (expt)), the theoretical value of the mass of the precursor molecule (Mr (calc)), the difference between the experimental value and the theoretical value of the mass of the precursor molecule (Delta), the number of uncleaved sites (Miss), the ion score (Score), the expected value (Expect), the score rank (Rank), being a unique peptide present only in a protein (Unique, described as “U” for a unique peptide), and the amino acid sequence of the peptide (Peptide). The symbol with “+DAABD (C)” displayed to the right of the symbol representing the amino acid sequence indicates that the Cys residue of the peptide is labeled with DAABD.

In the search results shown in FIG. 3, it was shown that for many peaks, each peptide was identified with a high ion score value, and all of these were attributed to human albumin (trypsin-digested fragment thereof). In addition, 20 peptides among the peptides shown to belong to human albumin were labeled with DAABD, and it was shown that in 18 peptides among these 20 peptides, a peak appeared at the position where the mass-to-charge ratio (m/z) was 303.06. The mass of the ion (C₁₀H₁₅NO₃S₂⁺) represented by the following structural formula (2) that can be generated by cleavage of a chemical bond from the structure in which DAABD-Cl is modified to a Cys residue is 303.06, and thus it can be said that the peak appearing at the position where the mass-to-charge ratio (m/z) is 303.06 is derived from the structure in which DAABD-Cl is modified to a Cys residue.

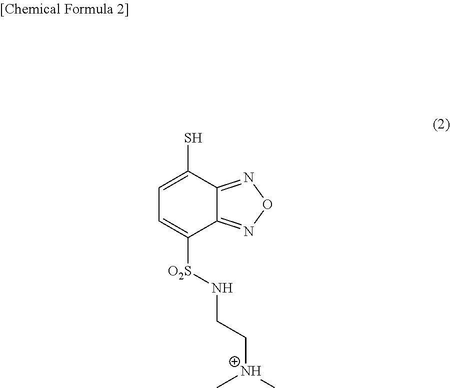 [SMILES: C[NH+](C)CCNS(=O)(=O)c1ccc(S)c2nonc12]

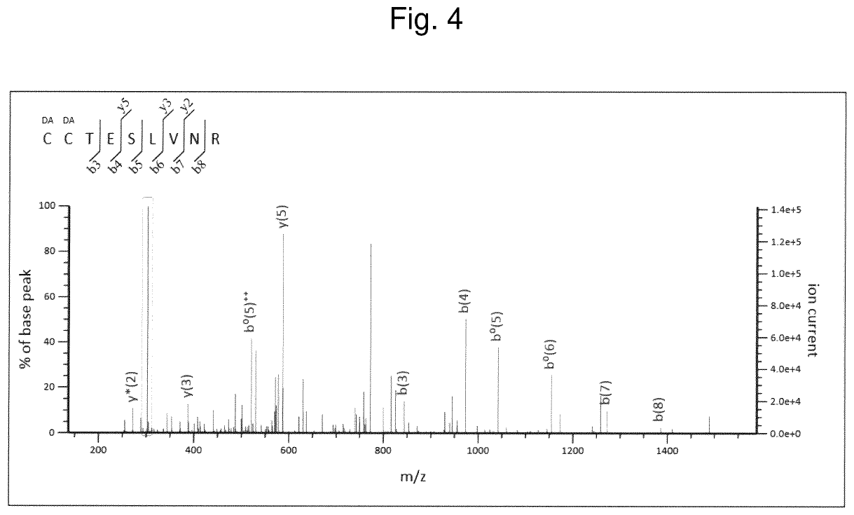

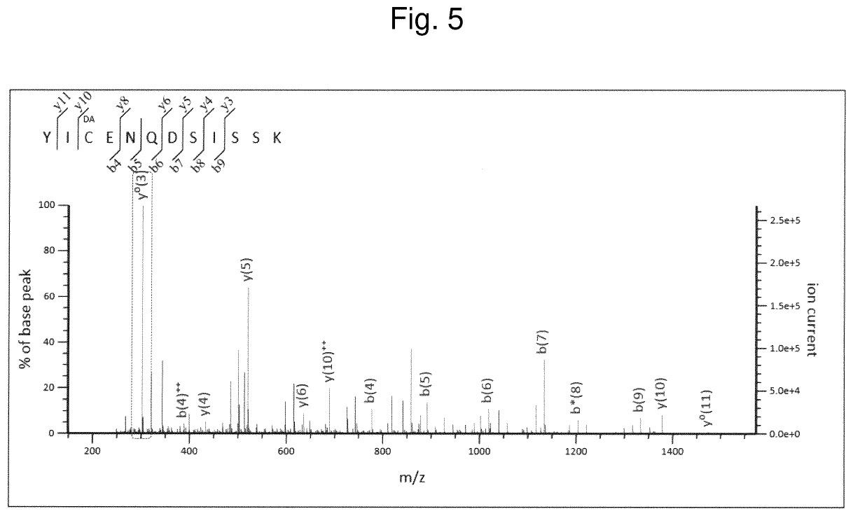

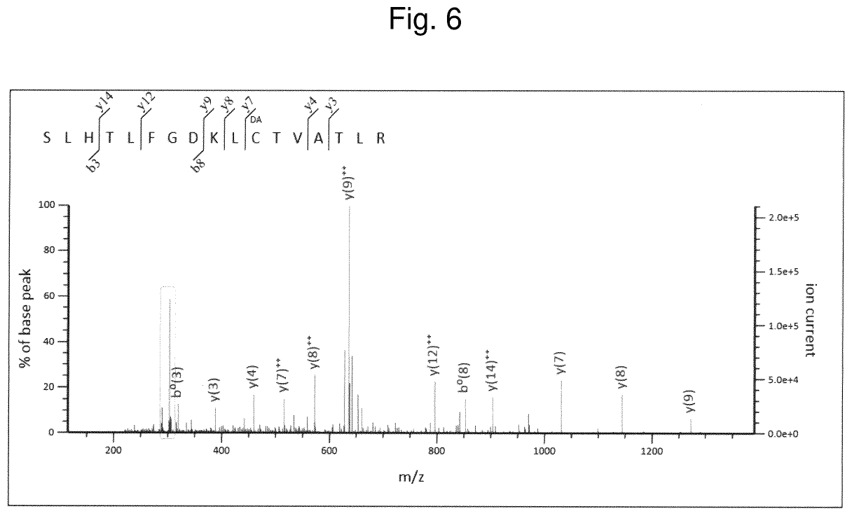

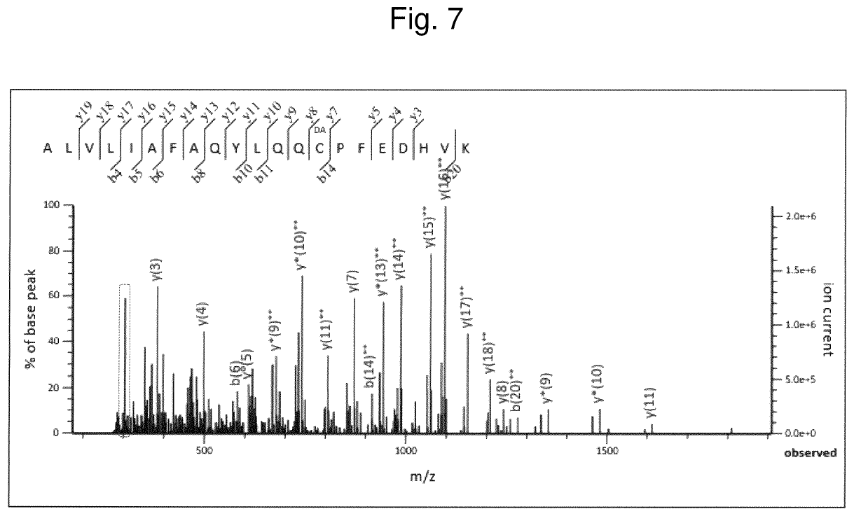

FIGS. 4 to 7 show MS/MS spectra of four types of peptides surrounded by a square frame in FIG. 3, and amino acid sequences of peptides estimated from the spectra. FIG. 4 shows a MS/MS spectrum of a peptide with serial number “571” (amino acid sequence: C*C*TESLVNR), FIG. 5 shows a MS/MS spectrum of a peptide with serial number “589” (amino acid sequence: YIC*ENQDSISSK), FIG. 6 shows a MS/MS spectrum of a peptide with serial number (Query) 627 (amino acid sequence: SLHTLFGDKLC*TVATLR), and FIG. 7 shows a MS/MS spectrum of a peptide with serial number (Query) 655 (amino acid sequence: ALVLIAFAQYLQQC*PFEDHVK) (C* indicates DAABD-labeled Cys). As can be seen from FIGS. 4 to 7, in the MS/MS spectra of these four peptides, a peak (in FIGS. 4 to 7, a peak surrounded by a square frame) was observed at the position where the mass-to-charge ratio (m/z) was 303.06, and all of the peptides contained a Cys residue. The peptide shown in FIG. 7 is a peptide containing Cys34 of human albumin.

From the above results, it was suggested that whether or not a peak appears at the position where the mass-to-charge ratio (m/z) on the MS/MS spectrum is 303.06 is an index for determining whether or not the peptide estimated from the MS/MS spectrum contains a reduced Cys residue in the original sample.

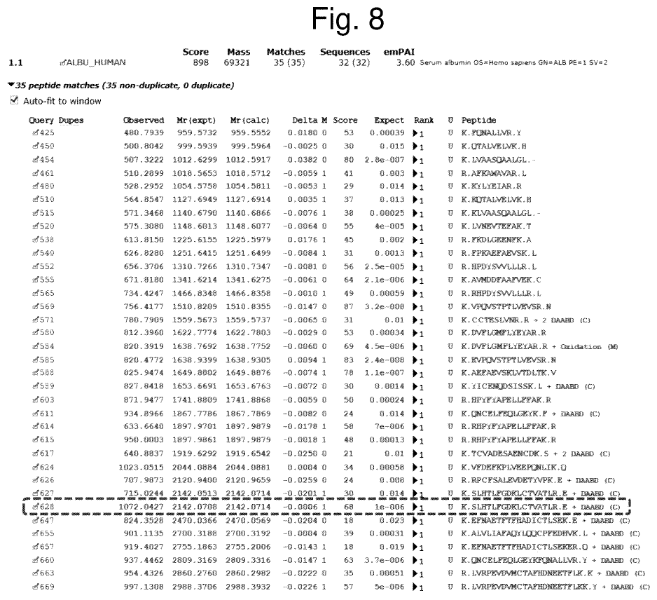

Although the reduced Cys residue of human albumin was labeled with DAABD, some MS/MS spectra did not show a peak at the position where the mass-to-charge ratio (m/z) was 303.06. It is presumed that such a peptide was not trapped because the ion score was low, or the mass-to-charge ratio of the precursor ions was close to or equal to or less than the cut-off value of the ion trap. FIG. 8 shows an example of the result of MS/MS ion search of such a peptide (in FIG. 8, peptides with serial numbers (Query) of 628 surrounded by a broken-line rectangular frame).

2. Measurement Example of Sample Containing Peptide Other than Albumin

DAABD-Cl was added to a sample containing glutathione and a sample containing oxytocin in the same manner as in the measurement example of the sample containing human albumin described above (with the exception of the step of trypsin digestion), and DAABD was bound to the Cys residue contained in each of glutathione and oxytocin. Glutathione is a tripeptide composed of glutamic acid, cysteine, and glycine (Glu-Cys-Gly). Oxytocin is a peptide composed of nine amino acids (Cys-Tyr-Ile-Gln-Asn-Cys-Pro-Leu-Gly).

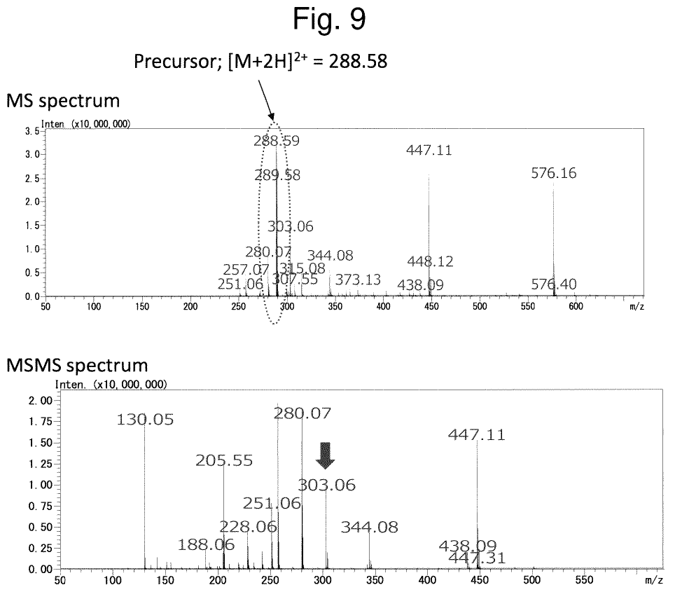

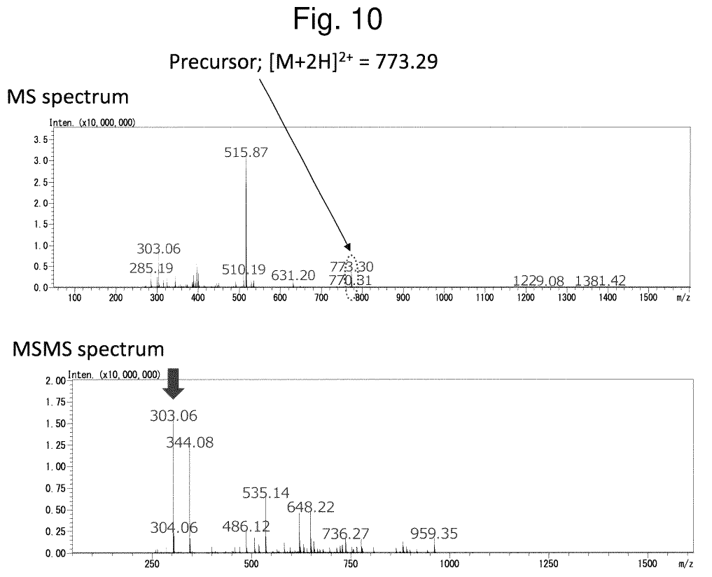

FIGS. 9 and 10 show the results of LC-MS/MS analysis of a sample containing glutathione and a sample containing oxytocin. The upper parts of FIG. 9 and FIG. 10 show the MS spectra, and the lower parts show the MS/MS spectra of precursor ions surrounded by an ellipse in the upper parts of the MS spectra.

As shown in the MS/MS spectra of FIGS. 9 and 10, it was confirmed that even in the sample containing glutathione and oxytocin, a peak was observed at the position where the mass-to-charge ratio (m/z) was 303.06.

### 3. Others

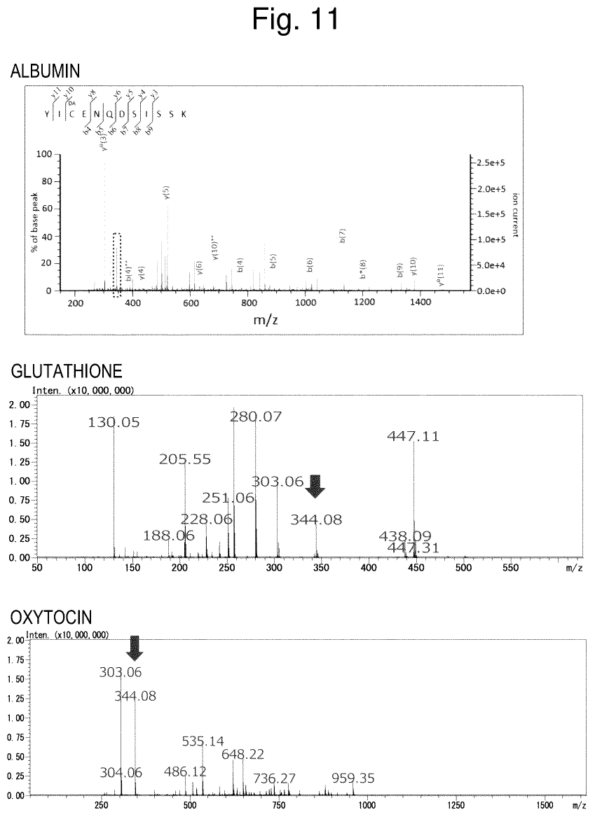

As shown in FIG. 11, it was found that a peak having a mass-to-charge ratio (m/z) of 344.08 commonly appeared on the MS/MS spectra obtained for each of the sample containing human albumin, the sample containing glutathione, and the sample containing oxytocin, in addition to the peak having a mass-to-charge ratio (m/z) of 303.06.

When the structure of an ion derived from a structure in which DAABD-Cl is modified to a Cys residue having a mass of 344.08 was examined, a structure represented by the following formula (3) was found. This suggests that similarly to the peak having a mass-to-charge ratio (m/z) of 303.06, the peak having a mass-to-charge ratio (m/z) of 344.08 is also a peak corresponding to the structure of DAABD-Cl, and may be an index for determining whether or not the Cys residue is in a reduced form.

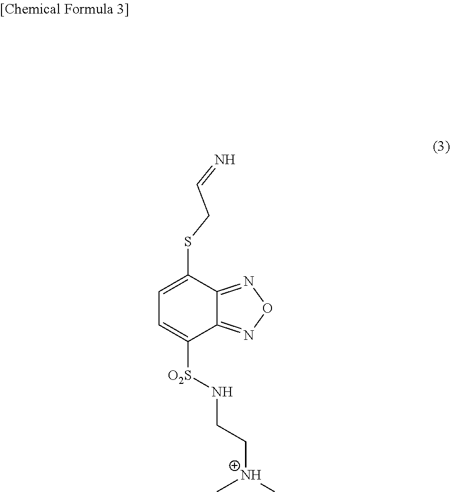 [SMILES: C[NH+](C)CCNS(=O)(=O)c1ccc(SCC=N)c2nonc12]

The present invention is not limited to the above described embodiments, and can be changed as appropriate.

For example, the SH group is not limited to the SH groups possessed by the Cys residue, and it is possible to determine whether or not the SH group is in a free state or forms an SS bond as long as the SH group is contained in a protein or peptide.

An experiment was performed by taking DAABD-Cl as an example of a labeling compound which specifically reacts with an SH group of a Cys residue contained in a protein or a peptide, but in addition to this, for example, a compound represented by the following general formula (4) or (5) or an isotope compound thereof, which are listed in Patent Literatures 1 and 2, can be used.

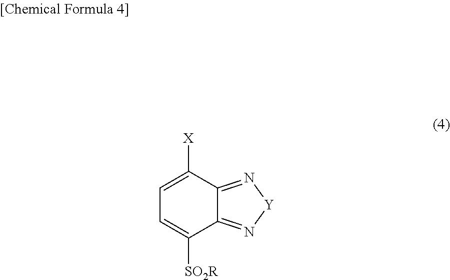

In the formula (4), X represents halogen, Y represents O, Se or S, and R represents —NH₂, —NHR′ (where R′ is an alkyl-substituted N-alkyl, a dialkyl-substituted N-alkyl, or a trialkyl-substituted N-alkyl), or —NR″R′″ (where R″ represents alkyl, R′″ represents alkyl-substituted N-alkyl, dialkyl-substituted N-alkyl, or trialkyl-substituted N-alkyl).

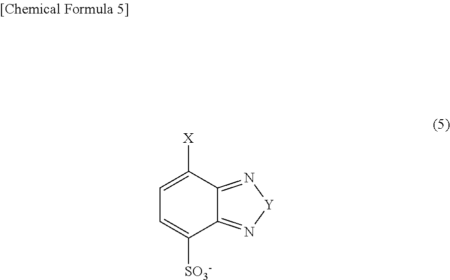

In the formula (5), X represents halogen, and Y represents O, Se, or S.

In the present invention, instead of the product ion scan measurement, there may be performed precursor ion scan measurement using ions, as product ions, derived from a functional group formed by a reaction between a thiol group desorbed from a Cys residue and a labeling compound, or with respect to the precursor ions used in the product ion scan measurement, there may be performed MRM measurement using a plurality of types of ions, as product ions, derived from a functional group formed by a reaction between a thiol group desorbed from a Cys residue and a labeling compound.

### REFERENCE SIGNS LIST

1 . . . LC Unit
2 . . . MS/MS Unit
3 . . . Analysis Controller
31 . . . Central Controller
32 . . . Input Unit
33 . . . Display Unit
34 . . . Cys
35 . . . Protein/Peptide Structure Estimation Control Program
4 . . . Data Processor
41 . . . Data Collection Unit
42 . . . Data Storage Unit
43 . . . Graph Creation Unit
44 . . . Peptide Structure Estimation Processing Unit
45 . . . Protein/Peptide Database

1. A peptide analysis method for analyzing a reduced form and an oxidized form of a cysteine residue contained in a peptide with a chromatograph mass spectrometer capable of MS/MS measurement, the peptide analysis method comprising:
a) a step of preparing a sample containing a peptide to be analyzed;
b) a step of adding a labeling compound which specifically reacts with a thiol group to the sample, and labeling a reduced cysteine residue contained in the peptide in the sample with the labeling compound;
c) a separation step of separating a sample containing the peptide labeled with the labeling compound by a chromatograph;
d) a measurement execution step of executing product ion scan measurement with a peptide ion, as a precursor ion, generated by ionizing the separated peptide labeled with the labeling compound under a condition that a thiol group is not desorbed from a cysteine residue; and
e) a determination step of determining whether or not the peptide to be analyzed contains a reduced cysteine residue by determining whether or not a peak appearing at a specific mass-to-charge ratio corresponding to a structure of the labeling compound is present in a mass spectrum created based on data obtained in the measurement execution step,
wherein the labeling compound is a compound represented by the following formula (1):

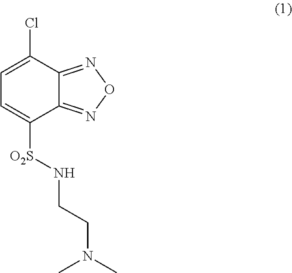 [SMILES: CN(C)CCNS(=O)(=O)c1ccc(Cl)c2nonc12]

2. The peptide analysis method according to claim 1, wherein
in the separation step, a sample obtained by fragmenting a peptide labeled with the labeling compound with a protein digestion enzyme is temporally separated by a chromatograph.

3. The peptide analysis method according to claim 1, wherein
a mass-to-charge ratio of a peak derived from a structure of the labeling compound is 303.06.

4. The peptide analysis method according to claim 1, wherein
a mass-to-charge ratio of a peak derived from a structure of the labeling compound is 344.08.

5. A peptide analysis apparatus comprising a chromatograph mass spectrometer capable of MS/MS measurement and configured to analyze a reduced form and an oxidized form of a cysteine residue contained in a peptide, the peptide analysis apparatus comprising:
a) an analysis controller configured to operate a chromatograph mass spectrometer so as to separate a sample containing a peptide labeled with a labeling compound which specifically reacts with a thiol group by a chromatograph, and to perform product ion scan measurement with a peptide ion, as a precursor ion, generated by ionizing the separated sample under a condition that a thiol group is not desorbed from a cysteine residue;
b) a mass spectrum creation unit configured to create a mass spectrum based on data obtained by the product ion scan measurement; and
c) a determination unit configured to determine whether or not a peak appearing at a specific mass-to-charge ratio corresponding to a structure of the labeling compound is present in the mass spectrum to determine whether or not a peptide to be analyzed contains a reduced cysteine residue,
wherein the labeling compound is a compound represented by the following formula (1): 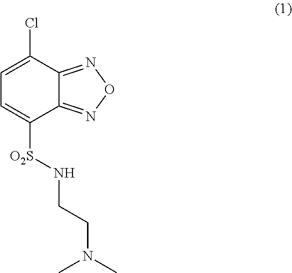 [SMILES: CN(C)CCNS(=O)(=O)c1ccc(Cl)c2nonc12]
